(19) United States
(12) Patent Application Publication
(10) Pub. No.: US 2005/0010169 A1
(43) Pub. Date: Jan. 13, 2005

(54) Medical apparatus for administering or evacuating a fluid to or from a human or animal body or for receiving such a fluid

(76) Inventors: Georg Kuhlein, Foslau (DE); Andreas Zeitler, Grafengehaig (DE)

Correspondence Address: VENABLE, BAETJER, HOWARD AND CIVILETTI, LLP, P.O. BOX 34385, WASHINGTON, DC, 20043-9998, US

(21) Appl. No.: 10/490,769

(22) PCT Filed: Sep. 24, 2002
(86) PCT No.: PCT/EP02/10680

(30) Foreign Application Priority Data
Sep. 26, 2001 (DE) 101 47 597.7

(51) Int. Cl. A61M 31/00

(52) U.S. Cl. 604/93.01; 427/2.1

(57) ABSTRACT

The invention relates to a medical apparatus for administering or evacuating a fluid to or from a human or animal body or for receiving said fluid, wherein the surface of the apparatus facing the fluid is structured in a knob-like manner by means of bumps whose measurements lie in the μm range.

The invention relates to a medical apparatus for administering or evacuating a fluid to or from a human or animal body or for receiving such a fluid.

For the treatment of a patient, in particular before, during and after an operation, fluid is frequently administered to or evacuated from the patient, for example blood, nutrient solutions and other dispersions or suspensions which are relevant for therapeutic or other reasons. Catheters or tubular lines through which the fluid is guided are generally used for this. These lines as a rule take the form of thin plastic tubing. In particularly when these lines are used over a longer period of time, as is frequently the case following operations or the like, bacteria will often settle in the line, which can lead to serious infections. It is also conceivable that these lines will clog up or close up over time, for example as a result of agglomerated blood or the like. To prevent fluid components or other particles from adhering to the inside of the line, attempts have been made to make the inside walls of this tubing fluid-repelling, in particular hydrophobic. The fluid-repelling characteristic is intended to prevent, for example, that blood platelets or fibrin or the like adhere to the inside wall. To produce hydrophobic plastic tubing, an additive that is incompatible with PVC is added to the tubing material, for example produced from PVC. Depending on the storage time and the storage temperature, this additive migrates to the surface on the line inside where it forms a micro-crystalline, fluid-repelling layer. The additive is so-to-speak “sweated out.”

Even though a certain improvement of the problem can be achieved in this way, the initially mentioned difficulties nevertheless occur quite frequently.

Another aspect is that the additives can be washed out and can themselves lead to difficulties due to possible incompatibility reactions or the like.

It is therefore the object of the present invention to provide a medical apparatus that prevents for the most part the adherence of micro-particles, e.g. in the form of bacteria, materials that create a favorable medium for bacteria and the like, so that the above-mentioned danger of infection is removed, especially if the instrument is used over a longer period of time.

This object is solved according to the invention for a medical apparatus of the above-mentioned type in that the apparatus surface facing the fluid is structured in a knob-like manner with bumps that are in the μm range.

The micro-structured knob-like surface according to the invention is particularly useful for preventing in most cases the adherence of micro-particles, such as bacteria or the like, so that the aforementioned problems are mostly avoided, particularly in view of the infection danger to the patient or the removal of the fluid itself. A reduced coagulation tendency has furthermore been observed during the contact with blood and thus an improved bio-compatibility. For this, the invention makes use of the so-called “Lotus Effect” which was originally discovered in the field of botany. The bumps provided according to the invention, which only measure a few μm and are positioned close together, prevent the adherence of micro-particles. In contrast to smooth surfaces to which particles can adhere relatively easily, the rough surface has so-to-speak self-cleaning characteristics. For the medical use, it means that this self-cleaning characteristic of a fluid line or a container or the like represents a spectacular improvement with respect to avoiding infections, but also with respect to patient comfort since catheters, for example, can be used for a longer period of time and an uncomfortable replacement frequently can be avoided.

Even though the micro-structuring itself already results in the self-cleaning characteristic because of the bumps in the μm range, it is advantageous if at least the bumps and preferably the complete inside of the line is fluid-repelling, in particular hydrophobic. This can be achieved, for example, by using fluid-repelling materials, preferably corresponding polymers, when producing the line or through subsequent hydrophobing, e.g. silanization of the plastic tubing, which should in particular have a silanol group density of 1-10 and preferably 2-6.

It is advantageous if the bumps are essentially lens-shaped, meaning they have a slightly convex form. The height of the bumps should range from 5-50 μm, in particular 20 μm, and the width and diameter of these bumps should also be in the same range.

It is particularly advantageous if the outside of the line is also structured in a knob-like manner by means of bumps in the μm range. This has the advantage of preventing in particular any micro-particles from adhering to the outside of the tubing and/or to permit an easy cleaning of the outside as well.

According to a first embodiment of the invention, the bumps can also be provided or created directly on the surface of the inside and, if necessary, also on the outside, meaning these bumps are formed as part of the plastic material itself. It is furthermore conceivable to create these bumps through micro-replication. Alternatively, a coating can also be applied to the inside and, if necessary, to the outside which forms the structured surface after it hardens, wherein a lacquer containing micro-particles is advantageously used for this. The material for the apparatus advantageously is a plastic material. Depending on the type of use, the plastic materials used can be polymers selected from a wide stiffness range, extending from soft-elastic to hard and tough, such as silicons, polyurethanes, polyolefins, polyamides, polystyrenes, PVCs and their co-polymers.

The apparatus itself can take the form of tubular lines, for example forming part of a catheter or the catheter itself, of a short and if necessary rigid line section that may take the form of a line connection or coupling section, or of a container, for example an infusion container or the like.

In addition to the medical apparatus itself, the invention furthermore also relates to the use of a medical apparatus of the above-described type for administering or evacuating a fluid to or from a human or animal body or for receiving such a fluid. The fluid can be blood or a watery solution, suspension or dispersion which is administered or evacuated or received.

The invention furthermore relates in general to a plastic material for medical uses, said material having an improved anti-bacterial effect when its surface comes in contact with a fluid required for a medical application. This plastic material distinguishes itself in that the surface which comes in contact with the fluid is structured in a knob-like manner by means of bumps with dimensions in the μm range. Advantageous further modifications of the plastic material according to the invention are disclosed in the dependent claims.

Additional advantages, features and details of the invention follow from the exemplary embodiment described below as well as from the drawings. Shown are in:

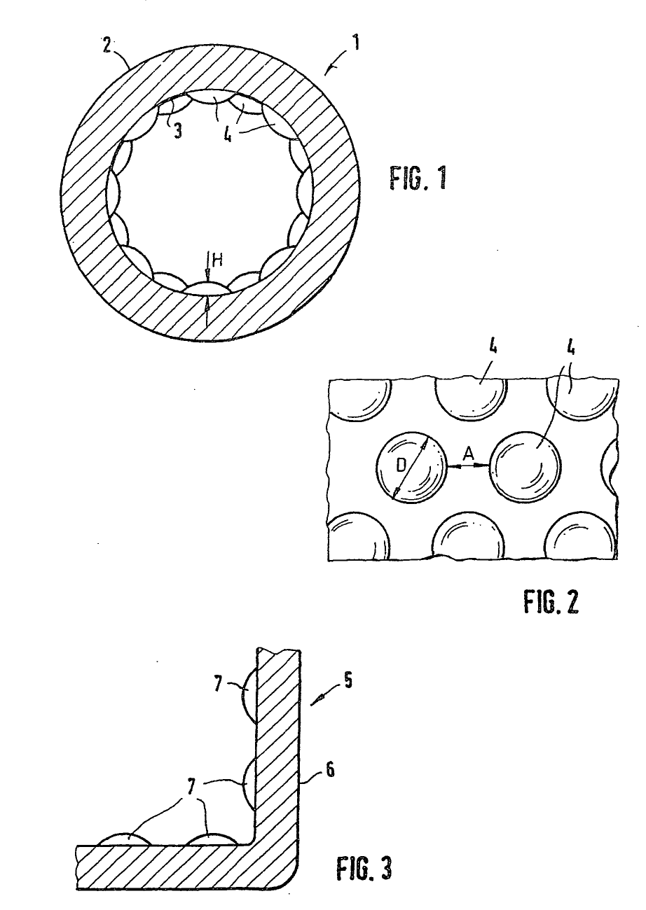

FIG. 1A basic sketch of a medical apparatus according to the invention, showing a sectional view of a tubular line;

FIG. 2A view from above of an unwound section of the tubing shown in FIG. 1, and

FIG. 3A partial view of a medical apparatus according to the invention in the form of a container.

FIG. 1 shows a medical apparatus 1 according to the invention, in a first embodiment in the form of a fluid line 2. This fluid line 2 has a tubular shape and is made from plastic. Knob-like bumps 4, which can be produced in an optional manner, are provided on the inside wall 3 of line 2 which comes in contact with the fluid. These knob-like bumps are distributed either in a regular arrangement or an irregular arrangement over the complete length of the line. They are essentially lens-shaped, meaning they have a convex surface, wherein the height H should be approximately 20 μm and the diameter D (see FIG. 2) should also be around 20 μm. The distance A between two bumps 4 should also be in the μm range, wherein distances between 1-100 μm are conceivable.

This knob-type structuring of the surface and/or the surface area coming in contact with the fluid provides the surface with a self-cleaning characteristic. The structuring prevents the adherence of micro-particles carried along in the fluid, e.g. blood platelets or fibrin or the like, or other particles present in a watery carrier medium and used for a medical application. The convex upper surface of the bumps 4 should be fluid-repelling, wherein of course the complete surface, meaning also the intermediate spaces between the bumps 4, can also be fluid-repelling.

FIG. 3 contains a basic sketch, showing a detail of another apparatus 5 according to the invention in the form of a container 6, of which only the lower edge region is shown. The inside of container 6 that is facing the fluid in this case is also provided with bumps 7 which have the same design as the bumps 4 shown for the exemplary embodiment in FIGS. 1 and/or 2. The bumps in this case are also distributed either in a regular specific pattern or an irregular arrangement across the inside surface of container 6. The adherence of micro-particles contained in a fluid inside the container 6— which is placed originally into the container or collected therein—is avoided by simply moving the fluid frequently, thereby causing the particles to be washed off the container inside wall by the fluid itself.

Of course, the same also applies to the fluid flowing through the line 2. Particles are washed off in this case as well, provided such particles have actually been deposited on the wall, for example if the fluid has not moved for a specific period of time.

Optional polymers can be used for the plastic material, depending on the stiffness required for the respective application and/or the medical apparatus that is produced. The medical apparatuses can be an optional plastic object, for example tubular lines, containers and the like.

1. A medical apparatus for administering or evacuating a fluid to or from a human or animal body or for receiving such a fluid,
characterized in that
the apparatus surface facing the fluid is structured in a knob-like manner by means of bumps in the μm range.

2. The medical apparatus according to claim 1, characterized in that at least the bumps, preferably the complete inside surface of the line is fluid-repelling, in particular hydrophobic.

3. The medical apparatus according to claim 1, characterized in that the bumps are essentially lens-shaped.

4. The medical apparatus according to claim 1, characterized in that the height of the bumps ranges from 5-5 μm and in particular is 20 μm.

5. The medical apparatus according to claim 1, characterized in that the width or the diameter of the bumps ranges from 5-50 μm and in particular is 20 μm.

6. The medical apparatus according to claim 1, characterized in that the outer surface and/or the surface of the apparatus which does not come in contact with the fluid is structured in a knob-like manner with bumps having dimensions in the μm range.

7. The medical apparatus according to claim 1, characterized in that the bumps are provided or created directly on the respective surface itself.

8. The medical apparatus according to claim 1, characterized in that the surface is created with the aid of a coating that contains the bumps and is structured once it hardens.

9. The medical apparatus according to claim 8, characterized in that the coating is a lacquer containing micro-particles.

10. The medical apparatus according to claim 1, characterized in that it consists of a plastic material.

11. The medical apparatus according to claim 1, characterized in that it is embodied as tubular line, as short and if necessary rigid line section or as container.

12. The use of a medical apparatus according to claim 1, for administering or evacuating a fluid to or from a human or animal body or for receiving such a fluid.

13. The use according to claim 12, characterized in that the fluid administered or evacuated or received is blood or a watery solution, suspension or dispersion.

14. A plastic material used for medical purposes with improved anti-bacterial effect when its surface comes in contact with a fluid necessary for a medical application, characterized in that the surface coming in contact with the fluid is structured in a knob-like manner with bumps in the μm range.

15. The plastic material according to claim 14, characterized in that at least the bumps and preferably the complete surface is fluid-repelling, in particular hydrophobic.

16. The plastic material according to claim 14, characterized in that the bumps essentially are lens-shaped.

17. The plastic material according to claim 14, characterized in that the height of the bumps is between 5-50 μm, in particular 20 μm.

18. The plastic material according to claim 14, characterized in that the width or the diameter of the bumps ranges from 5-50 μm, in particular is 20 μm.

19. The plastic material according to claim 14, characterized in that even the surface which does not come in contact with the fluid is structured in a knob-like manner with bumps in the μn range.

20. The plastic material according to claim 14, characterized in that the bumps are created or provided directly on the surface itself.

21. The plastic material according to claim 14, characterized in that a coating is deposited on the surface which is structured once it hardens.

22. The plastic material according to claim 21, characterized in that a lacquer containing micro-particles is deposited as the coating.

23. The plastic material according to claim 14, selected from the group containing silicons, polyurethanes, polyolefins, polyamides, polystyrenes, PVCs or their co-polymers.
